(19) United States
(12) Patent Application Publication
(10) Pub. No.: US 2004/0142388 A1
(43) Pub. Date: Jul. 22, 2004

(54) Method for detecting chronic dementia diseases, and corresponding VGF peptides and detection reagents

(76) Inventors: Norbert Lamping, Hannover (DE); Hans-Dieter Zucht, Hannover (DE); Michael Jurgens, Hannover (DE); Gabriele Heine, Hannover (DE); Rudiger Hess, Hannover (DE); Markus Kellmann, Harpstedt (DE); Hartmut Selle, Hannover (DE)

Correspondence Address: Whitham, Curtis & Christofferson, PC, Suite 340, 11491 Sunset Hills Road, Reston, VA, 20190, US

(21) Appl. No.: 10/680,087

(22) Filed: Oct. 6, 2003

Related U.S. Application Data
(63) Continuation-in-part of application No. PCT/DE02/01376, filed on Apr. 8, 2002.

(30) Foreign Application Priority Data
Apr. 6, 2001 (DE) 101 17 431.4

(51) Int. Cl. G01N 33/53; G01N 33/567

(52) U.S. Cl. 435/7.2

(57) ABSTRACT

The invention relates to defined peptides and the quantitative determination thereof in biological samples from patient's suffering from Alzheimer's disease, in relation to the concentration thereof in a control group. The invention also relates to the use of said peptides for therapeutic purposes. The inventive peptides come from a protein precursor having the corresponding gene and are processed in a specific manner and modified in a post-translation manner. Changes in the concentrations of said peptides indicate Alzheimer's disease, and the direction of the change in concentration is specifie for each peptide. Alzheimer's disease is detected by identifying the peptides individually or in groups. The invention can also be used to control the course of Alzheimer's disease, for the prognosis thereof and for the development of therapeutic agents to combat the same.

This is a continuation-in-part (CIP) application of International Application PCT/DE02/01376 with an international filing date of Apr. 8, 2002,now abandoned.

### FIELD OF THE INVENTION

The invention relates to a method for detecting a chronic dementia disease or a predisposition to a chronic dementia disease, in particular Alzheimer's disease or related neurological diseases, e.g. Lewy body dementia or vascular dementia. The invention further relates to peptides which have been found for detecting the presence of these diseases, for monitoring the course of the diseases and of the grade of the diseases. In addition, the invention relates to detection reagents such as antibodies and nucleic acids and the like, via which these peptides or the corresponding nucleic acids can be detected. The invention further relates to pharmaceutical applications which comprise VGF, VGF peptides, VGF antibodies, VGF nucleic acids, VGF protein antagonists, VGF protein agonists, VGF peptide agonists or VGF peptide antagonists for the therapy or prophylaxis of neurological diseases, especially of Alzheimer's disease. The invention further relates to methods for identifying patients with neurological diseases, especially Alzheimer's disease, who are suitable for taking part in clinical studies to investigate these diseases.

The peptides comprise fragments of the VGF protein, which is also called neuroendocrine specific protein VGF. The abbreviation VGF is also used in the literature for the protein “vaccinia growth factor” or for “vaccinia virus growth factor” and for “vascular permeability factor”, these proteins not corresponding to the VGF protein to which the invention relates.

### BACKGROUND OF THE INVENTION

Dementia diseases represent an increasing problem in industrialized countries because of the higher average life expectancy. Dementia diseases are in most cases incurable and make long-term care of the patients necessary. About half of these patients receive inpatient care. More than 60 dementia diseases are known, including diseases associated with manifestations of dementia.

However, Alzheimer's disease (AD) accounts for about 65% of these, and the diagnosis and therapy thereof is therefore of great importance. Besides Alzheimer's disease, the following non-Alzheimer's dementias are known, inter alia: vascular dementia, Lewy body dementia, Binswanger dementia, and dementia diseases which occur as concomitant effects of other disorders such as Parkinson's disease, Huntington's disease, Pick's disease, Gerstmann-Sträussler-Scheinger disease, Kreuzfeldt-Jakob disease etc.

Alzheimer's disease is a neurodegenerative disease distinguished by the following symptoms: decline in intellectual abilities, confusion and diminished ability to look after themselves. A greatly restricted short-term memory in particular is characteristic of Alzheimer's disease, whereas the patient's memories of the distant past, e.g. of his/her own childhood, is impaired far less by the disease. There are morphological changes in the brain manifested inter alia in the form of amyloid deposits and degenerated nerve cells. The morphological changes can be diagnosed histologically after the patient's death and are as yet the only reliable detection of the disease. These histopathological diagnoses are based on criteria fixed by the Consortium to Establish a Registry for Alzheimer's Disease (CERAD). The following criteria-based diagnostic systems are currently used to diagnose Alzheimer's disease: the International classification of Diseases, 10th revision (ICD-10), the Diagnostic and Statistical Manual of Mental Disorders, 4th edition (DSM-IV) of the American Psychiatric Association, and the Work Group crieria drawn up by the National Institute of Neurological and Communicative Disorders Association NINCDS-ADRDA.

These systems use a number of neuropsychological tests in order to diagnose Alzheimer's disease, but not objectively measurable clinical parameters.

Diagnosis of Alzheimer's disease is also difficult because it, just like the other dementia diseases, has an insidious onset and is associated with slowly progressive destruction of nerve cells in the brain.

At present, no causal therapy is available for the treatment of Alzheimer's disease. The disease is merely treated symptomatically, e.g. by administration of neurotransmitters such as acetylcholine. Further possible therapeutic strategies being tested at present are the administration of antioxidants, of radical scavengers, of calcium channel blockers, of antiinflammatory substances, of secretase inhibitors, of anti-amyloid antibodies etc., and immunization against amyloid peptides. However, no causal therapy of this disease is yet possible.

### SUMMARY OF THE INVENTION

The invention is based on the object of avoiding the prior art disadvantages in the diagnosis of Alzheimer's disease and of providing a method which can be used early and reliably for detecting chronic dementia diseases, especially Alzheimer's disease. It is additionally based on the object of providing a novel therapy for the treatment of Alzheimer's disease because, at present, only unsatisfactory therapeutic approaches to the treatment of Alzheimer's disease are available.

### DESCRIPTION OF THE DRAWINGS

FIG. 1: Alignment of the VGFARP peptides with the two known VGF proteins, corresponding to the database accession No. NM_{—}003378 and Y12661, e.g. Seq. IDs 43 and 44

FIG. 2: Reverse phase chromatography for separation and enrichment of VGFARP peptides from cerebrospinal fluid

FIG. 3: Mass spectrometric measurement (MALDI) on VGFARP-7 (SEQ ID NO:7) as example

FIG. 4: MALDI as relatively quantifying mass spectroscopic method

FIG. 5: MS/MS fragment spectrum of the peptide VGFARP-13 (SEQ ID NO:11) as example

FIGS. 6a:-C: Box-whisker plots for quantitative comparison of the concentrations of VGFARP-1(SEQ ID NO:1), VGFARP-2(SEQ ID NO:2), VGFARP-18(SEQ ID NO:15), VGFARP-3(SEQ ID NO:3), VGFARP-4(SEQ ID NO:4), VGFARP-5(SEQ ID NO:5), VGFARP-6(SEQ ID NO:6), VGFARP-7(SEQ ID NO:7), VGFARP-19(SEQ ID NO:16), VGFARP-20(SEQ ID NO:17), VGFARP-21(SEQ ID NO:18), VGFARP-10(SEQ ID NO:8), VGFARP-22(SEQ ID NO:19), VGFARP-28(SEQ ID NO:25), VGFARP-29(SEQ ID NO:26), VGFARP-30/32(SEQ ID NO:27/SEQ ID NO:29), VGFARP-31(SEQ ID NO:28), VGFARP-12 (SEQ ID NO:10), VGFARP-13 (SEQ ID NO:11), VGFARP-36 (SEQ ID NO:33), VGFARP-37 (SEQ ID NO:34), VGFARP-40(SEQ ID NO:37), VGFARP-41 (SEQ ID NO:38) and VGFARP-42 (SEQ ID NO:39) in Alzheimer's disease patients compared with control patients.

### DETAILED DESCRIPTION OF THE INVENTION

Definitions:

VGF Proteins or Peptides (SEQ ID NOS:44 and 43) Corresponding to Accession Nos. NM-003378 and Y12661: (SEQ ID NOS:46 and 45, Respectively)

The peptides (SEQ ID NOS:43 and 44) derived from the nucleic acid sequences NM-003378 and Y12661 (SEQ ID NOS:44 and 43, respectively) are also referred to as VGF proteins and include all naturally occurring alleles, mutants and polymorphisms of VGF proteins, and tissue-specifically expressed VGF variants. Included in particular are also the VGF variants which occur because of diseases or as a result of neurological diseases, especially chronic dementia diseases, especially Alzheimer's disease. There is inclusion both of VGF proteins with and without signal sequence, proforms of VGF proteins which have not yet been processed, and already processed VGF proteins, soluble VGF proteins and membrane-associated VGF proteins, where the membrane-associated VGF proteins may be linked both via transmembrane amino acid sequences to a cell membrane or organelle membrane and via a post-translational modification, e.g. a glycosyl-phosphatidyl-inositol (GPI) anchor. Also included are variations of the VGF sequence which [lacuna] by alternative splicing, by alternative translation starting and termination points, by RNA editing, by alternative post-translational modifications, and other VGF protein variants arising through naturally occurring mechanisms.

VGFARP Peptides:

VGF peptides and VGF peptide variants are referred to hereinafter as VGFARP (VGF Alzheimer related peptide) peptides. VGFARP peptides may be derived from both the VGF sequences mentioned at the outset (NM_{—}003378=Seq. ID 43 for the protein and Seq. ID 45 for the DNA) and Y1266=Seq. ID 44 for the protein and Seq. ID 46 for the DNA) and from other VGF protein variants possibly occurring in nature. In addition, VGFARP peptides may include two point-mutated, two deleted and/or two additionally internally inserted amino acids, and/or N-terminal and/or C-terminal extensions. However, in these cases they must retain at least 8 amino acids from the VGF protein sequence. VGFARP-39 (SEQ ID NO:36) is an exception from this rule, as VGFARP-39 (SEQ ID NO:36) has only a legth of 6 amino acids. The only amino acids suitable as N- or C-terminal extensions are those occurring in the VGF protein sequence at this sequence position in the VGF protein. Peptides derived from naturally occurring VGF polymorphisms and from naturally occurring VGF mutants are also referred to as VGFARP peptides. VGFARP peptides may also exist with post-translational modifications such as, for example, glycosylations and phosphorylations and/or in chemically modified form, preferably as peptide oxides. For example, VGFARP-12 (SEQ ID NO:10) has been identified both as non-oxidized and as oxidized peptide.

Chemically or Post-Translationally Modified Peptides:

A chemically or post-translationally modified peptide may consist both of D- and of L-amino acids, and of combinations of D- and L-amino acids. These peptides may additionally comprise unusual amino acids, i.e. amino acids which do not belong to the 20 standard amino acids. Examples of unusual amino acids are, inter alia: alpha-aminobutyric acid, beta-aminobutyric acid, beta-alanine, beta-aminoisobutyric acid, norvaline, homoserine, norleucine, gamma-aminobutyric acid, thioproline, 4-hydroxyproline, alpha-aminoadipic acid, diaminobutyric acid, 4-aminobenzoic acid, homocysteine, alpha-aminopenicillanic acid, histamine, ornithine, glycine-proline dipeptide, hydroxylysine, proline-hydroxyproline dipeptide, cystathionine, ethionine, seleno-cysteine. Possible post-translational or chemical modifications are, inter alia, modifications of amino acid sequences by the following structures: linkage of free cysteine to a cysteine in the peptide sequence, methyl, acetyl, farnesyl, biotinyl, stearoyl, palmityl, lipoyl, C-mannosyl, phosphorus and sulfate groups, glycosylations, amidations, deamidations, pyroglutamic acid, citrulline etc.

Nucleic Acids:

Nucleic acids are regarded as being DNA, RNA and DNA-RNA hybrid molecules both of natural origin and prepared synthetically or by recombination. Also included are chemically modified nucleic acids which comprise modified nucleotides having high in vivo stability, such as, for example, phosphorothioates. Such stabilized nucleic acids are already used in the application of ribozyme, antisense and triplex nucleic acid techniques.

Significance:

The term significant is used in the sense in which the term significance is used in statistics. In this patent application, an error probability of less than 90%, preferably 95% further preferably 99% is defined as significant.

Sensitivity:

Sensitivity is defined as the proportion of patients with the disease who acquire a positive diagnostic result in a diagnosis for the disease, i.e. the diagnosis correctly indicates the disease.

Specificity:

The specificity is defined as the proportion of healthy patients who acquire a negative diagnostic result in a diagnosis for the disease, i.e. the diagnosis correctly indicates that no disease is present.

It has surprisingly been found that only in samples of body fluids from patients suffering from Alzheimer's disease, especially in the cerebrospinal fluid, is the concentration of certain peptides changed greatly relative to their concentration in control samples, and thus makes detection of Alzheimer's disease possible. Changes in the concentration of these peptides relative to their concentration in control groups indicate the presence of Alzheimer's disease and are therefore suitable for detecting this disease with high sensitivity and specificity. Modulation of the VGF protein or VGFARP peptide concentration with the aim of adjusting the patient to normal VGF or VGFARP levels can thus be used therapeutically.

To achieve the object, the invention includes a method for detection of a neurological, in particular of a chronic dementia disease, in particular of Alzheimer's disease, or of a predisposition to such a disease by identifying one or more VGF peptides which are derived from the sequence having the Gene Bank accession No. NM_{—}003378 or the accession No. Y12661 of the DNA Data Bank of Japan (Seq. ID 43 or 44), in a biological sample from an individual. Since these VGF peptides are presumably causally connected with the disease, the present invention also includes the use of these peptides for the therapy of Alzheimer's disease or related neurological diseases. These peptides or peptide fragments are referred to as VGF derived Alzheimer related peptides (VGFARP). The two VGF protein variants NM_{—}003378 and Y12661 (SEQ ID NOS:44 and 43, respectively) differ only at 13 positions of their amino acid sequence and VGF peptides which make it possible to distinguish between Alzheimer's disease and the control group have been identified from both VGF proteins. The VGFARP peptides VGFARP-11 (SEQ ID NO:9), 32 (SEQ ID NO:29) and -44 (SEQ ID NO:41) are derived from the VGF variant with the accession No. Y12661(SEQ ID NO:43), and the VGFARP peptides VGFARP-25(SEQ ID NO:22), -30(SEQ ID NO:27), -31(SEQ ID NO:28), -36(SEQ ID NO:33) and -37(SEQ ID NO:34) are derived from the VGF variant with the accession No. NM-_{—}003378(SEQ ID NO:44). All the other VGFARP peptides can be derived on the basis of their amino acid sequence from both of the two VGF variants. Since VGFARP peptides derived from two different variants have already been identified, it must be assumed that further VGFARP peptides derived from these or other VGF variants also exist. The invention likewise relates to these VGFARP peptides.

To achieve the object, the invention indicates a method for the detection of Alzheimer's disease by determination of the relative concentration of at least one marker peptide in a biological sample from a patient compared with the concentration of the marker peptide in a control sample, in which the following points must be satisfied: 1. At least one VGFARP peptide or a peptide that is derived from the nucleic acids with the accession Nos. NM_{—}003378 or Y12661 (Seq. IDs 45 and 46) or homologous sequences is used as marker peptide. 2. An increase or decrease specific for the particular marker peptide occurs in the concentration of the marker peptide in the patient's sample relative to the concentration of the marker peptide in the control sample. 3. A significant change in the concentration of the marker peptide in the aforementioned manner is regarded as a positive detection result for a neurological disease, preferably Alzheimer's disease.

In this connection, it is possible in principle for a particular VGFARP peptide either to undergo only an increase in the peptide concentration in Alzheimer's disease patients, or it is possible in principle for this VGFARP peptide to undergo only a reduction in the peptide concentration of Alzheimer's disease patients. For a defined VGFARP peptide it is not possible for the VGFARP peptide concentration simultaneously to be increased in one individual Alzheimer's disease patient and to be reduced, relative to the control group, in another Alzheimer's disease patient. As with virtually all medical diagnoses of diseases, false-positive or false-negative results are possible in principle, i.e. that in a few individual cases an incorrect diagnosis takes place because the concentration of the VGFARP peptides in Alzheimer's disease patients does not differ with hundred percent probability from the concentration of the VGFARP peptides in control samples. This problem can, however, be eliminated by multiple controls.

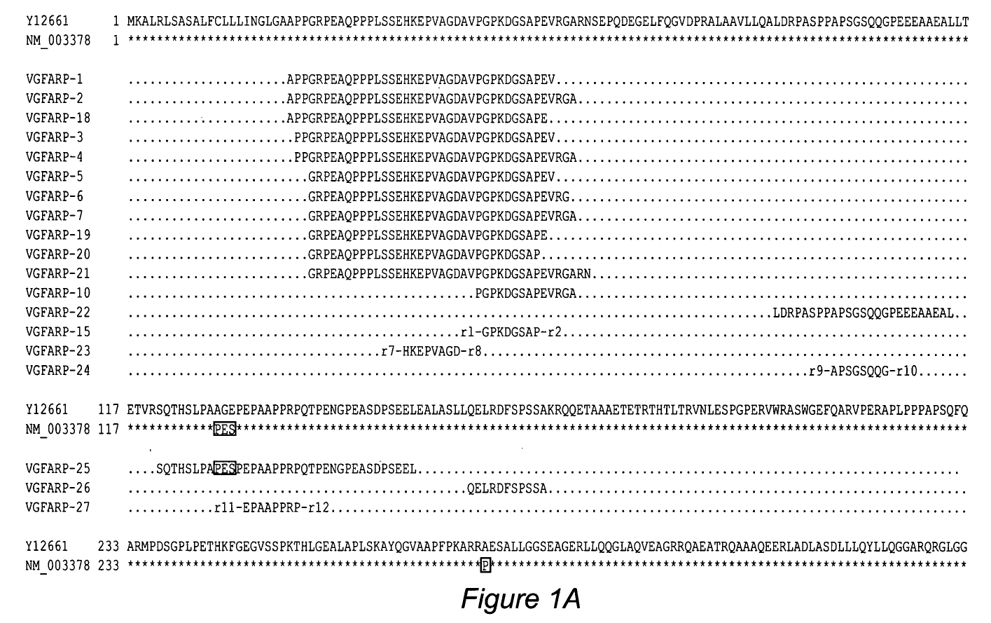

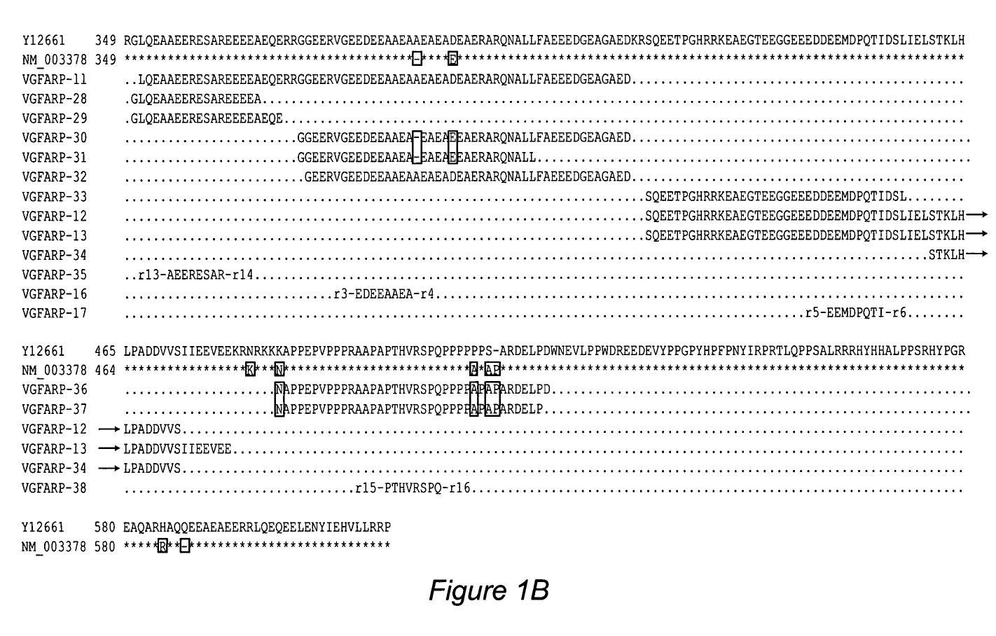

Peptides which can be regarded as fragments of the VGF sequence are referred to as VGFARP peptides for the purposes of this invention. They include homologous peptides derived from VGF. They include derivatives of naturally occurring alleles of these peptides and homologous mutants, especially point-mutated mutants with preferably not more than two amino acids differing from VGF. Preferred markers according to the invention are indicated in the sequence listing and thus named from VGFARP-1 (SEQ ID NO:1) to -7(SEQ ID NO:7), VGFARP-10 (SEQ ID NO:8) to -13 (SEQ ID NO:11) and VGFARP-15(SEQ ID NO:12) to -45(SEQ ID NO:42), corresponding to Seq. ID 1 to 42. The sequences of the VGFARP peptides are depicted in FIG. 1 and in Table 1. The assignment of the VGFARP peptides to their respective Seq. ID No. is shown in Table 1.

The method of the invention comprises a method in which there is measurement of specific biomarkers whose concentration is changed in neurodegenerative diseases, especially in Alzheimer's disease, and which indicate the disease even in a very early stage and indicate an increased risk of the disease at an early date. This is important in order to provide a reliable clinical marker for diagnosing these diseases.

It is possible and preferable for the concentration of VGFARP peptides in the sample, but also the characteristic pattern of occurrence of the plurality of particular VGFARP peptides, to be correlated with the severity of the disorder. These novel markers therefore make it possible to develop and monitor therapies for the treatment of Alzheimer's disease, because the course and any successful cure resulting from a therapy or a diminished progression of the disease can be established. Effective therapy of Alzheimer's disease is not possible at present, underlining the urgency for the provision of a reliable detection method for Alzheimer's disease, because reliable detection of the disease is a precondition for the development of a therapy.

Detection of VGFARP peptides additionally makes it possible in the framework of clinical studies to develop novel therapies for the treatment of Alzheimer's disease with high specificity to select only those patients suffering from Alzheimer's disease and not from other diseases. This is important for obtaining valid study results. Patients incorrectly diagnosed as Alzheimer's disease patients have a negative influence on the quality of the results of a study on Alzheimer's disease therapy. In addition, detection of VGFARP peptides makes it possible to stratify patients, i.e. the specific selection of subgroups of Alzheimer's disease patients who are especially suitable for particular Alzheimer's disease therapeutic strategies or clinical studies.

There are marked changes in the concentrations of VGFARP peptides in Alzheimer's disease patients relative to healthy people. A further aspect of the invention is therefore a bringing of the VGFARP concentrations in Alzheimer's disease patients to normal concentrations. This method can be employed for the therapy of Alzheimer's disease or related neurological diseases. If the VGF protein or VGFARP peptide concentrations are elevated, the concentrations of these substances can be reduced by therapeutic administration of, for example, VGF protein- or VGFARP peptide-specific antibodies or VGF-specific antisense nucleic acids, ribozymes or triplex nucleic acids for VGFARP peptide antagonists, VGF protein antagonists. Substances which suppress the endogenous expression of VGF protein or the processing of VGF protein to VGFARP peptides can also be administered for the therapy. If the disease is caused by a deficiency of VGF protein or VGFARP peptides, therapeutic doses of VGF protein, VGFARP peptides, VGFARP peptide agonists or VGF protein agonists can be given. Endogenous production of VGF protein or VGFARP peptides can be increased by therapeutic administration of substances such as, for example, NGF, BNDF or NT-3 or other suitable substances, because these substances increase VGF expression. Substances which promote the processing of VGF protein to VGFARP peptides such as, for example, prohormone convertases such as, for example, PC1, PC2 or PC3, can also be employed therapeutically. Combination of different therapeutic strategies is, of course, also possible and sensible in some circumstances.

The invention therefore also encompasses the use of VGF proteins, VGFARP peptides, VGFARP peptide agonists and antagonists, VGF protein agonists and antagonists, anti-VGF protein antibodies, anti-VGFARP peptide antibodies, NGF, BNDF, NT-3, anti-NGF antibodies, anti-BNDF antibodies, anti-NT-3 antibodies and antibodies against receptors of said proteins for the direct or indirect modulation of the concentration of the VGF proteins and VGFARP peptides for the treatment of neurological diseases, especially Alzheimer's disease. Alternative to antibodies, it is also possible to use antibody fragments, antibody fusion proteins, or other substances which bind selectively to VGF proteins, VGFARP peptides, NGF, BNDF or NT-3. It is also possible as alternative to said proteins and peptides for fusion proteins of said proteins to be used. The invention further encompasses also the use of antisense nucleic acids, triplex nucleic acids and ribozymes which modulate the expression of said proteins and peptides. The invention additionally encompasses agonists and antagonists which modulate the activity of said proteins.

A further embodiment of the invention is the pharmaceutical formulation or chemical modification of the described peptides and nucleic acids to make it possible for them to cross the blood-brain barrier and/or the blood-CSF barrier more efficiently. They are thus made particularly suitable for therapeutic use. In order to achieve this, it is possible for example for VGF peptides, VGF proteins, nucleic acids, agonists or antagonists to be modified so that for example they become more lipophilic, favoring entry into the subarachnoid space. This can be achieved by introducing hydrophobic molecular constituents or else by “packaging” the substances in hydrophobic agents, e.g. liposomes. It is additionally possible for example for peptide sequences to be attached to these peptides, proteins, nucleic acids, agonists or antagonists, which favor crossing into the subarachnoid space or, conversely, impede crossing out of the subarachnoid space.

The invention also encompasses the administration of said therapeutic agents by various routes such as, for example, as intravenous injection, as substance which can be administered orally, as inhalable gas or aerosol, or administration in the form of direct injection into the subarachnoid space, or into tissue such as muscle, fat, brain etc. It is possible in this way to achieve increased bioavailability and efficacy of these therapeutic agents. For example, peptides or proteins administered orally can be protected by acid-resistant capsules from proteolytic degradation in the stomach. Very hydrophobic substances can become more hydrophilic and thus better suited for, for example, intravenous injections by suitable pharmaceutical processing etc.

A further embodiment of the invention is the use of VGFARP peptides or of VGF proteins for identifying receptors which selectively bind these molecules. These receptors can also be modulated by administration of agonists or antagonists, which is expedient for the therapy of neurological diseases, especially of Alzheimer's disease.

Owing to the large number of VGF peptides newly identified within the framework of this invention, it is possible for the first time to detect experimentally positions in the VGF protein at which processing of the VGF protein takes place in vivo. These processing sites comprise, based on the VGF protein sequence of NM_{—}003378 (SEQ ID NO:44), the following sequence positions: 371/372, 418/419, 479/480, 480/481, 481/482, 482/483 and 483/484. Based on the VGF protein sequence of Y12661(SEQ ID NO:43), the processing sites are as follows: 371/372, 419/420, 480/481, 483/484, 484/485 and 485/486. All experimentally identified processing positions represent dibasic positions, i.e. directly consecutive amino acids having positively charged amino acid side chains (arginine=R, lysine=K). Such sequence motifs are recognized and cut for example by prohormone convertases, with additional endoproteolytic deletion of the two basic amino acids. As the name of the prohormone convertases indicates, prohormones are converted by prohormone convertases to hormones, resulting in new bioactive substances (peptide hormones). Examples of biological active peptides which are generated in this way from their proforms are proNGF/NGF, pro BDNF/BNDF etc. [1]. Consequently, the VGFARP peptides of the invention represent peptide hormones which are suitable in connection with neurological diseases, preferably Alzheimer's disease, as points of attack for therapeutic agents. Modulation of the VGFARP peptide concentrations can thus be used for the therapy of neurological diseases, preferably Alzheimer's disease.

VGF Biology

The VGF proteins (VGF peptide precursor molecules) identified within the framework of this invention are synthesized as proteins about 68 kDa in size selectively in neuroendocrine and neuronal cells, with expression thereof decreasing with increasing age [2]. Investigation of VGF gene-deficient mice revealed that important function in energy metabolism are affected [3]. VGF gene-deficient mice have a small body size, are hypermetabolic and hyperactive. VGF is also synthesized in the insulin-producing islet cells of the pancreas.

VGF was discovered on investigation of a rat pheochomozytome cell line (PC12 cell line), and stimulation of this cell line with “nerve growth factor” (NGF) brings about a 12- to 14-fold increase in the concentration of VGF [4, 5]. NGF is an important growth factor which regulates the differentiation of the peripheral and central nervous system. Further factors which regulate VGF expression are brain-derived neurotrophic factor (BDNF) and neurotropin-3 (NT-3) [6]. VGF mRNA is regulated in vivo by neuronal activity, neuronal injuries and by the biological rhythm (circadian clock) [2, 7-9].

VGF is proteolytically processed with increasing differentiation of neuronal cells via neuron-specifically expressed endoproteases, which presumably recognize basic amino acids. As Trani et al. were able to show, C-terminal VGF peptides with masses of 20, 18 and 10 kDa are produced [10]. This VGF processing takes place in the postendoplasmic reticulum. These peptides accumulate in secretory vesicles, are released preferably by membrane depolymerization and might possibly play a role in neuronal communications [10]. Prohormone convertases such as, for example, PC1, PC2 or PC3 are known from the literature as examples of endoproteases which proteolytically cleave protein precursor molecules at dibasic sequence sites. The VGFARP peptides identified by us are, however, surprisingly fragments with a distinctly lower molecular weight than 10 to 20 kDa, and are therefore different from the VGF peptides described by Trani et al. In addition, the anti-VGF antibodies used by Trani et al. to detect these VGF peptides recognize VGFARP peptides which are different from the sequences of the VGFARP peptides. We have detected VGFARP peptides both in Alzheimer's disease patients and in the control group. The peptides identified by us represent novel VGF processing products which have not previously been described. The concentrations of the VGFARP peptides may be either uniformly raised or else uniformly lowered, in a manner which is specific for each peptide, in the patient group relative to the control group. Exclusively other VGF peptides of unknown sequence, derived from the C-terminal region of the VGF protein and having a distinctly higher molecular weight than the peptides newly identified and sequenced for the first time by us, were previously known [10].

### PREFERED EMBODIMENTS OF THE INVENTION

The chronic dementia disease detected by the method of the invention is preferably Alzheimer's disease. It has been possible to date to detect the change in the concentration of the peptides and peptide fragments of the invention in Alzheimer's disease patients. It can be concluded from this that the peptides of the invention can be used for the detection and for the therapy of Alzheimer's disease and related neurological diseases.

The identification is preferably concentrated on particular peptide fragments of the VGF proteins having the GeneBank accession No. NM_{—}003378, or the DDBJ accession No. Y12661 (Seq. IDs 43 and 44), i.e. on peptides which comprise partial sequences of these VGF proteins. These VGF peptides (VGF protein fragments) are referred to as VGF derived Alzheimer related peptide (VGFARP) and they are represented by Seq. ID 1 to 42. The alignment of the VGF proteins and VGFARP peptides is depicted in FIG. 1. The sequences we found for the peptides are indicated in the sequence listing.

We have detected various VGF peptides derived from two VGF protein variants for the first time in biological samples. These peptides, which are referred to as VGFARP peptides, represent defined fragments of VGF proteins. These fragments are produced in a natural way in nature and have not previously been described in the literature. These fragments are different from peptides generated in the literature often by in vitro proteolysis (by addition of proteases such as, for example, trypsin). They therefore represent novel, previously unknown substances. These peptides were initially enriched and purified from biological samples by reverse phase chromatography and subsequently separated by mass spectrometry from other accompanying peptides, so that it was subsequently possible to sequence these VGFARP peptides.

TABLE 1
The sequences of the peptides in the
single-letter amino acid code are as follows:

| | | | | |
|---|---|---|---|---|
| | | | Monoisotop | |
| VGF-Sequenz | VGFARP | Seq. | theoret. | |
| Position | No. | ID | mass (Da) | Sequence |

| | | | | | |
|---|---|---|---|---|---|
| Y12661 | NM_003378 | | | | |
| 23-59 | 23-59 | 1 | 1 | 3666.8278 | APPGRPEAQPPPLSSEH |
| | | | | | KEPVAGDAVPGPKDGSA |
| | | | | | PEV |
| 23-62 | 23-62 | 2 | 2 | 3950.9875 | APPGRPEAQPPPLSSEH |
| | | | | | KEPVAGDAVPGPKDGSA |
| | | | | | PEVRGA |
| 23-58 | 23-58 | 18 | 15 | 3567.7594 | APPGRPEAQPPPLSSEH |
| | | | | | KEPVAGDAVPGPKDGSA |
| | | | | | PE |
| 24-59 | 24-59 | 3 | 3 | 3595.7907 | PPGRPEAQPPPLSSEHK |
| | | | | | EPVAGDAVPGPKDGSAP |
| | | | | | EV |
| 24-62 | 24-62 | 4 | 4 | 3879.9504 | PPGRPEAQPPPLSSEHK |
| | | | | | EPVAGDAVPGPKDGSAP |
| | | | | | EVRGA |
| 26-59 | 26-59 | 5 | 5 | 3401.6852 | GRPEAQPPPLSSEHKEP |
| | | | | | VAGDAVPGPKDGSAPEV |
| 26-61 | 26-61 | 6 | 6 | 3614.8077 | GRPEAQPPPLSSEHKEP |
| | | | | | VAGDAVPGPKDGSAPEV |
| | | | | | RG |
| 26-62 | 26-62 | 7 | 7 | 3685.8448 | GRPEAQPPPLSSEHKEP |
| | | | | | VAGDAVPGPKDGSAPEV |
| | | | | | RGA |
| 26-58 | 26-58 | 19 | 16 | 3302.6167 | GRPEAQPPPLSSEHKEP |
| | | | | | VAGDAVPGPKDGSAPE |
| 26-57 | 26-57 | 20 | 17 | 3173.5741 | GRPEAQPPPLSSEHKEP |
| | | | | | VAGDAVPGPKDGSAP |
| 26-64 | 26-64 | 21 | 18 | 3955.9889 | GRPEAQPPPLSSEHKEP |
| | | | | | VAGDAVPGPKDGSAPEV |
| | | | | | RGARN |
| 49-62 | 49-62 | 10 | 8 | 1336.6735 | PGPKDGSAPEVRGA |
| 90-114 | 90-114 | 22 | 19 | 2503.1827 | LDRPASPPAPSGSQQGP |
| | | | | | EEEAAEAL |
| *50ᵣ₁- | 50₊ᵣ₁-57₊ᵣ₂ | 15 | 12 | ≧727.3501 | r1-GPKDGSAP-r2 |
| 57₊ᵣ₂ | | | | | |
| 39-46 | 39-46 | 23 | 20 | 851.4137 | r7-HKEPVAGD-r8 |
| 50-57 | 98-105 | 24 | 21 | ≧730.3246 | r9-APSGSQQG-r10 |
| — | 121-156 | 25 | 22 | 3745.7343 | SQTHSLPAPESPEPAAP |
| | | | | | PRPQTPENGPEASDPSE |
| | | | | | EL |
| 164-174 | 164-174 | 26 | 23 | 1235.5782 | QELRDFSPSSA |
| 133₊ᵣ₁₁- | 133₊ᵣ₁₁ - | 27 | 24 | ≧833.4395 | r11-EPAAPPRP-r12 |
| 140₊ᵣ₁₂ | 140₊ᵣ₁₂ | | | | |
| 351-418 | — | 11 | 9 | 7518.2744 | LQEAAEERESAREEEEA |
| | | | | | EQERRGGEERVGEEDEE |
| | | | | | AAEAAEAEADEAERARQ |
| | | | | | NALLFAEEEDGEAGAED |
| 350-367 | 350-367 | 28 | 25 | 2031.8981 | GLQEAAEERESAREEEE |
| | | | | | A |
| 350-370 | 350-370 | 29 | 26 | 2418.0419 | GLQEAAEERESAREEEE |
| | | | | | AEQE |
| — | 373-417 | 30 | 27 | 4806.0408 | GGEERVGEEDEEAAEAE |
| | | | | | AEAEEAERARQNALLFA |
| | | | | | EEEDGEAGAED |
| — | 373-404 | 31 | 28 | 3456.5513 | GGEERVGEEDEEAAEAE |
| | | | | | AEAEEAERARQNALL |
| 374-418 | — | 32 | 29 | 4806.0408 | GEERVGEEDEEAAEAAE |
| | | | | | AEADEAERARQNALLFA |
| | | | | | EEEDGEAGAED |
| 421-456 | 420-455 | 33 | 30 | 4058.7043 | SQEETPGHRRKEAEGTE |
| | | | | | EGGEEEDDEEMDPQTID |
| | | | | | SL |
| ** | 420-471 | 12 | 10 | 5776.6294 | SQEETPGHRRKEAEGTE |
| 421-472 | | | | | EGGEEEDDEEMDPQTID |
| | | | | | SLIELSTKLHLPADDVV |
| | | | | | S |
| 421-479 | 420-478 | 13 | 11 | 6618.0363 | SQEETPGHRRKEAEGTE |
| | | | | | EGGEEEDDEEMDPQTID |
| | | | | | SLIELSTKLHLPADDVV |
| | | | | | SIIEEVEE |
| 460-472 | 459-471 | 34 | 31 | 1380.7249 | STKLHLPADDVVS |
| 355₊ᵣ₁₃- | 355₊ᵣ₁₃- | 35 | 32 | ≧946.4468 | r13-AEERESAR-r14 |
| 362₊ᵣ₁₄ | 362₊ᵣ₁₄ | | | | |
| 381₊ᵣ₃- | 381₊ᵣ₃- | 16 | 13 | ≧862.3192 | r3-EDEEAAEA-r4 |
| 388₊ᵣ₄ | 388₊ᵣ₄ | | | | |
| 446₊ᵣ₅- | 445₊ᵣ₅- | 17 | 14 | ≧961.4063 | r5-EEMDPQTI-r6 |
| 453₊ᵣ₆ | 452₊ᵣ₆ | | | | |
| — | 485-522 | 36 | 33 | 3903.0180 | NAPPEPVPPPRAAPAPT |
| | | | | | HVRSPQPPPPAPAPARD |
| | | | | | ELPD |
| — | 485-521 | 37 | 34 | 3787.9911 | NAPPEPVPPPRAAPAPT |
| | | | | | HVRSPQPPPPAPAPARD |
| | | | | | ELP |
| 501₊ᵣ₁₅- | 500₊ᵣ₁₅- | 38 | 35 | ≧920,4828 | r15-PTHVRSPQ-r16 |
| 508₊ᵣ₁₆ | 507₊ᵣ₁₆ | | | | |
| 26-31 | 26-31 | 39 | 36 | 656.3242 | GRPEAQ |
| 25-62 | 25-62 | 40 | 37 | 3782.8976 | PGRPEAQPPPLSSEHKE |
| | | | | | PVAGDAVPGPKDGSAPE |
| | | | | | VRGA |
| 177-193 | 177-193 | 41 | 38 | 1886.8970 | QQETAAAETETRTHTLT |
| 177-191 | 177-191 | 42 | 39 | 1672.7653 | QQETAAAETETRTHTLT |
| 180-187 | 180-187 | 43 | 40 | ≧792.3501 | r17-TAAAETET-r18 |
| 374-404 | — | 44 | 41 | 3343.4672 | GEERVGEEDEEAAEAAE |
| | | | | | AEADEAERARQNAL |
| 457-476 | 456-475 | 45 | 42 | 2220.1889 | IELSTKLHLPADDVVSI |
| | | | | | IEE |

| Y12661 - Protein | 43 | Complete VGF-protein sequence |
|---|---|---|
| | | deduced from Y12661 of the |
| | | DNA Data Bank of Japan |
| NM_003378 - Protein | 44 | Complete VFG-protein sequence |
| | | decuced from NM_003378 of the |
| | | NCBI Data Bank |
| Y12661 - DNA | 45 | Complete VGF-DNA sequence |
| | | from DNA Data Bank of Japan |
| NM_003378 - DNA | 46 | Complete VGF-DNA sequence |
| | | from NCBI Data Bank |
| * r1 represents a sequence which corresponds to the sequence or parts of the sequence of the VGF protein from amino acid 49-23, and r1 can be between 0 and 27 amino acids long, starting from amino acid 50 of the VGF protein. Correspondingly, r2 represents the VGF | | |
| # protein sequence from amino acid 58 to 64 or parts thereof, and r2 can be between 0 and 7 amino acids long, starting from VGF amino acid 57. r3 represents the VGF protein sequence from amino acid 380 to 373 or parts therof, r4 represents the VGF protein sequence | | |
| # from amino acid 389 to 418 or parts thereof, r5 represents the VGF sequence from amino acid 445 to 421 or parts thereof, r6 represents the VGF protein sequence from amino acid 454 to 479 of parts thereof, r7 represents the VGF protein sequence from amino acid 38 | | |
| # to 23 of parts thereof, r8 represents the VGF protein sequence from amino acid 47 to 64 or parts thereof, r9 represents the VGF protein sequence from amino acid 97 to 90 or parts thereof, r10 represents the VGF protein sequence from amino acid 106 to 114 or | | |
| # parts thereof, r11 represents the VGF protein sequence from amino acid 132 to 121 or parts thereof, r12 represents the VGF protein sequence from amino acid 141 to 156 or parts thereof, r13 represents the VGF protein sequence from amino acid 354 to 350 or parts thereof, | | |
| # r14 represents the VGF protein sequence from amino acid 363 to 370 or parts thereof, r15 represents the VGF protein sequence from amino acid 500 to 486 or parts thereof, r16 represents the VGF protein sequence from amino acid 509 to 523 or parts thereof, r17 represents | | |
| # the VGF protein sequence from amino acid 179 to 177 or parts thereof, r18 represents the VGF protein sequence from amino acid 192 to 193 or parts thereof. | | |

** VGFARP-12 was identified as nonoxidized and as monooxidized peptide (increase in the molecular weight by about 16 dalton).

Suitable Peptides

The peptides can exist in post-translational or chemical modification forms, thus influencing inter alia their masses and the identification by mass spectrometry and also the eluation behavior on chromatography such as, for example, on reverse phase chromatography. In particular, the peptides may be in glycosylated, phosphorylated, sulfated, amidated, oxidized etc. form in the sample to be investigated. The modified peptides are preferably in the form of peptide oxide such as, for example, the peptide VGFARP-12 which was identified both as unmodified peptide and as peptide oxide.

The peptides are also regarded as VGFARP peptides in particular when individual amino acids differ from the corresponding sequence of the VGF protein, in particular when a maximum of 2 amino acids differ from the VGF protein sequence. It is permissible in this connection for there to be point mutations, deletions, internal insertions of amino acids, and N- and C-terminal extensions, as long as the VGFARP peptide sequence comprises at least 8 amino acids which are conserved, i.e. unchanged, relative to the amino acid sequence of the relevant VGF protein. VGFARP-39 represents an exception, as it only contains 6 amino acids.

For a positive detection of the disease, it is furthermore provided in a further development of the invention for the concentration of the identified peptide(s) to be raised or lowered for each of these peptides in a specific manner relative to the concentration of the respective peptide in a control sample. The ratio of the concentrations of the respective peptides to the concentration of the control sample can be used to determine the severity of the disease.

The control sample may be a pooled sample from various controls. The sample to be investigated may also be a pooled sample, and where there is a positive result individual investigations are subsequently carried out.

Suitable Biological Samples

The biological sample may preferably be cerebrospinal fluid (CSF) or a sample such as serum, plasma, urine, stool, tear fluid, synovial fluid, sputum etc. This depends inter alia on the sensitivity of the chosen detection method (mass spectrometry, ELISA etc.). It is also possible where appropriate to use homogenized tissue samples, tissue sections and biopsy specimens. It is therefore provided in a further embodiment of this invention for tissue homogenates to be produced, for example from human tissue samples obtained in biopsies, for preparation of the sample to be investigated. These tissues can be comminuted for example with manual homogenizers, with ultrasound homogenizers or with electrically operated homogenizers such as, for example, Ultraturrax, and then be boiled in a manner known to the skilled worker in acidic aqueous solutions with, for example, 0.1 to 0.2 M acetic acid for 10 minutes. The extracts are then subjected to the respective detection method, e.g. a mass spectrometric investigation. The samples can be prepared, for example where appropriate diluted or concentrated, and stored in the usual way.

Use of the VGFARP Peptides for Producing Diagnostic Agents

The invention further comprises the use of at least one VGFARP peptide of the invention or of a VGF protein for the diagnosis of neurological diseases, especially chronic dementia diseases, especially of Alzheimer's disease, and the use of VGFARP peptides for obtaining antibodies or other agents which, because of their VGFARP peptide-specific binding properties, are suitable for developing diagnostic reagents for detecting these diseases. The invention also encompasses the use of VGFARP peptides for obtaining phage particles which bind these peptides specifically, or which conversely present VGFARP peptides on their surface and thus make it possible to identify binding partners such as, for example, receptors of VGF proteins or VGFARP peptides.

Detection Methods for the VGFARP Peptides

Various methods can be used for detecting the VGFARP peptides within the framework of the invention. Methods suitable are those which make it possible to detect VGFARP peptides specifically in a patient's sample. Suitable methods are, inter alia, physical methods such as, for example, mass spectrometry or liquid chromatography, molecular biology methods such as, for example, reverse transcriptase polymerase chain reaction (RT-PCR) or immunological detection techniques such as, for example, enzyme linked immunosorbent assays (ELISA).

Physical Detection Methods

One embodiment of the invention is the use of physical methods which are able to indicate the peptides of the invention qualitatively or quantitatively. These methods include, inter alia, mass spectrometry, liquid chromatography, thin-layer chromatography, NMR (nuclear magnetic resonance) spectroscopy etc. This entails comparison of quantitative measured results from a sample to be investigated with the measurements obtained in a group of patients suffering from neurological diseases, in particular chronic dementia diseases, preferably Alzheimer's disease, and a control group. It is possible to infer the presence of a neurological diseases, in particular a chronic dementia disease, in particular Alzheimer's disease, and/or the severity of this disease from these results.

In a preferred embodiment of this invention, the peptides in the sample are separated by chromatography before the identification, in particular preferably by reverse phase chromatography, with particular preference for separation of the peptides in the sample by high-resolution reverse phase high performance chromatography (RP-HPLC). A further embodiment of this invention is the carrying out of precipitation reactions to fractionate the sample using precipitants such as, for example, ammonium sulfate, polyethylene glycol, trichloroacetic acid, acetone, ethanol etc. The fractions obtained in this way are subjected singly to the respective detection method, e.g. the investigation using mass spectrometry. A further embodiment of the invention is the use of liquid phase extraction. For this purpose, the sample is mixed with a mixture of an organic solvent such as, for example, polyethylene glycol (PEG) and an aqueous salt solution. Owing to their physical properties, particular constituents of the sample then accumulate in the organic phase, and others in the aqueous phase, and can thus be separated from one another and subsequently analyzed further.

Reverse Phase Chromatography

A particularly preferred embodiment of this invention encompasses the use of reverse phase chromatography, in particular a C18 reverse phase chromatography column using mobile phases consisting of trifluoroacetic acid and acetonitrile, for separation of peptides in human cerebrospinal fluid. For example the fractions collected in each case each comprise 1/100 of the mobile phase volume used. The fractions obtained in this way are analyzed with the aid of a MALDI mass spectrometer (matrix-assisted laser desorption ionization) using a matix solution consisting of, for example, of L(-) fucose and alpha-cyano-4-hydroxycinnamic acid dissolved in a mixture of acetonitrile, water, trifluoroacetic acid and acetone, and thus the presence of particular masses is established and the signal intensity quantified. These masses correspond to the masses of the VGFARP peptides of the invention.

Mass Spectrometry

In a preferred embodiment of the invention, VGFARP peptides can be identified with the aid of mass spectrometric determination, preferably a MALDI (matrix-assisted laser desorption and ionization) mass spectrometry. In this case, the mass spectrometric determination further preferably includes at least one of the following mass signals, in each case calculated on the basis of the theoretical monoisotopic mass of the corresponding peptide. It is possible for slight differences from the theoretical monoisotopic mass to show owing to the experimental error and the natural isotope distribution. In addition, in MALDI mass determinations a proton is added to the peptides owing to the method of measurement, whereby the mass increases by 1 dalton. The following masses correspond to the theoretical monoisotopic masses of the peptides identified by us; calculated with suitable software, in this case GPMAW 4.02. These theoretical monoisotopic masses may occur singly or in combination in a sample: VGFARP-1 (SEQ ID NO:1) =3666.8278/VGFARP-2 (SEQ ID NO:2)=3950.9875/VGFARP-18 (SEQ ID NO:15)=3567.7594/VGFARP-3 (SEQ ID NO:3)=3595.7907/VGFARP-4 (SEQ ID NO:4)=3879.9504/VGFARP-5 (SEQ ID NO:5)=3401.6852/VGFARP-6 (SEQ ID NO:6)=3614.8077/VGFARP-7 (SEQ ID NO:7)=3685.8448/VGFARP-19 (SEQ ID NO:16)=3302.6167/VGFARP-20 (SEQ ID NO:17)=3173.5741/VGFARP-21 (SEQ ID NO:18)=3955.9889/VGFARP-10 (SEQ ID NO:8)=1336.6735/VGFARP-22 (SEQ ID NO:19)=2503.1827/VGFARP-15 (SEQ ID NO:12)=≧727.3501/VGFARP-23 (SEQ ID NO:20)=≧851.4137/VGFARP-24 (SEQ ID NO:21)=≧730.3246/VGFARP-25 (SEQ ID NO:22)=3745.7343/VGFARP-26 (SEQ ID NO:23)=1235.5782/VGFARP-27 (SEQ ID NO:24)=≧833.4395/VGFARP-11 (SEQ ID NO:9)=7518.2744/VGFARP-28 (SEQ ID NO:25)=2031.8981/VGFARP-29 (SEQ ID NO:26)=2418.0419/VGFARP-30 (SEQ ID NO:27) 4806.0408/VGFARP-31 (SEQ ID NO:28)=3456.5513/VGFARP-32 (SEQ ID NO:29)=4806.0408/VGFARP-33 (SEQ ID NO:30)=4058.7043/VGFARP-12 (SEQ ID NO:10)=5776.6294/VGFARP-13 (SEQ ID NO:11)=6618.0363/VGFARP-34 (SEQ ID NO:31)=1380.7249/VGFARP-35 (SEQ ID NO:32)=≧946.4468/VGFARP-16 (SEQ ID NO:13)=≧862.3192/VGFARP-17 (SEQ ID NO:14)=≧961.4063/VGFARP-36 (SEQ ID NO:33)=3903.0180/VGFARP-37 (SEQ ID NO:34)=3787.9911/VGFARP-38 (SEQ ID NO:35)=≧920.4828/VGFARP-39 (SEQ ID NO:36)=656.3242/VGFARP-40 (SEQ ID NO:37)=3782.8976/VGFARP-41 (SEQ ID NO:38)=1886.8970/VGFARP-42 (SEQ ID NO:39)=1672.7653/VGFARP-43 (SEQ ID NO:40)=≧792.3501/VGFARP-44 (SEQ ID NO:41)=3343.4672 and VGFARP-45 (SEQ ID NO:42)=2220.1889.

The symbol=≧ (is greater than or equal to) is to be understood to mean that the relevant VGFARP peptides cannot have any larger masses but can have only the masses possible owing to the amino acids which are possibly additionally present at the ends of these peptides. Amino acids which may be additionally present at the ends of these peptides are not just any ones but only those which may be present at this sequence position owing to the sequence of the VGF protein.

Mass Spectrometric Determination of the Sequence of the VGFARP Peptides

For the further practical application of this embodiment, further confirmation of the result of detection is advisable and possible by establishing the identity of the peptides corresponding to the masses, taking account exclusively of peptide signals which may be derived from a VGF protein. This confirmation takes place by identifying the peptide signals preferably using methods of mass spectrometry, e.g. MS/MS analysis [11].

Novel, specific peptides of VGF proteins (VGFARP peptides) were identified, and their significance was revealed by the method of the invention. These peptides and their derivatives are referred to herein as VGFARP peptides. Their sequences are indicated in the sequence listing. The VGFARP peptides VGFARP-15 (SEQ ID NO:12), 16 (SEQ ID NO:13), -17 (SEQ ID NO:14), -27 (SEQ ID NO:24), -35 (SEQ ID NO:32), 38 (SEQ ID NO:35) and VGFARP-43 (SEQ ID NO:40) may comprise on the N- and/or C-terminus additional amino acids corresponding to the corresponding sequence of the relevant VGF protein. The invention also encompasses the VGFARP peptides prepared recombinantly or synthetically, and isolated from biological samples, in unmodified, chemically modified or post-translationally modified form. In this connection, two point mutations and other differences are possible as long as the VGFARP peptide has at least 8 amino acids which agree in their identity and their position within the peptide sequence with a VGF protein.

Molecular Biology Detection Techniques

Finally, the invention also encompasses nucleic acids which correspond to VGFARP peptides, and especially those which correspond to the VGFARP peptides of the invention, the use thereof for the indirect determination and quantification of the relevant VGF proteins and peptides. This also includes nucleic acids which represent, for example, noncoding sequences such as, for example, 5′- or 3′-untranslated regions of the mRNA, or nucleic acids which show a sequence agreement with the VGF nucleic acid sequence which is sufficient for specific hybridization experiments and which are therefore suitable for the indirect detection of relevant proteins, especially the VGFARP peptides.

One exemplary embodiment thereof encompasses the obtaining of tissue samples, e.g. of biopsy specimens, from patients and the subsequent determination of the concentration of an RNA transcript corresponding to the gene having the GeneBank accession No. NM_{—}03378 or the accession No. Y12661 of the DNA Data Bank of Japan, DDBJ or corresponding to homologous VGF variants. This entails comparison of quantitative measured results (intensities) from a sample to be investigated with the measurements obtained in a group of patients suffering from Alzheimer's disease and a control group. Methods which can be used for the quantification are, for example, reverse transcriptase polymerase chain reaction (RT-PCR), quantitative real-time PCR (ABI PRISM® 7700 Sequence Detection System, Applied Biosystems, Foster City, Calif., USA), in situ hybridization or Northern blots in a manner known to the skilled worker. The presence of a chronic dementia disease, preferably Alzheimer's disease and/or the severity thereof can be inferred from the results.

Immunological Detection Methods

In a further preferred embodiment of the invention, the VGFARP peptides or the VGF proteins can be identified using an immunological detection system, preferably an ELISA (enzyme linked immuno sorbent assay). This immunological detection picks up at least one VGFARP peptide or VGF protein. To increase the specificity, it is also possible and preferred to use the so-called sandwich ELISA in which the detection of the VGFARP peptides depends on the specificity of two antibodies which recognize different epitopes within the same molecule. However, it is also possible to use other ELISA systems, e.g. direct or competitive ELISA, to detect VGFARP peptides or VGF proteins. Other ELISA-like detection techniques such as, for example, RIA (radio immuno assay), EIA (enzyme immuno assay), ELI-Spot etc. are also suitable as immunological detection systems. VGFARP peptides or VGF proteins isolated from biological samples, recombinantly prepared or chemically synthesized can be used as standard for the quantification. Identification of the VGFARP peptide(s) is generally possible for example with the aid of an antibody directed to the VGFARP peptide or VGF protein. Further methods suitable for such detections are, inter alia, Western blotting, immunoprecipitation, Dot-Blots, plasmon resonance spectrometry (BIACORE®-Technologie, Biacore International AB, Uppsala, Sweden), phage particles, PNAs (peptide nucleic acids), affinity matrices (e.g. ABICAP-Technologie, ABION Gesellschaft fur Biowissenschaften und Technik mbH, Jülich, Germany) etc. Substances/molecules suitable as detection agents are generally all those permitting the construction of a specific detection system because they specifically bind a VGFARP peptide or VGF protein.

Obtaining of VGFARP Peptides and Anti-VGFARP Peptide Antibodies

A further embodiment of the invention is the obtaining of VGFARP peptides using recombinant expression systems, chromatographic methods and chemical synthesis protocols which are known to the skilled worker. The VGFARP peptides obtained in this way can be used inter alia as standards for quantifying the respective VGFARP peptides or as antigen for producing VGFARP peptide antibodies. Methods known to the skilled worker and suitable for isolating and obtaining VGFARP peptides include the recombinant expression of peptides. It is possible to use for the expression of the VGFARP peptides inter alia cell systems such as, for example, bacteria such as Escherichia coli, yeast cells such as Saccharomyces cerevisiae, insect cells such as, for example, Spodoptera frugiperda (Sf-9) cells, or mammalian cells such as Chinese Hamster Ovary (CHO) cells. These cells are obtainable from the American Tissue Culture Collection (ATCC). For recombinant expression of VGFARP peptides, for example nucleic acid sequences which code for VGFARP peptides are inserted in combination with suitable regulatory nucleic acid sequences such as, for example, promoters, antibiotic selection markers etc. into an expression vector by molecular biology methods. A vector suitable for this purpose is, for example, the vector pcDNA3.1 from Invitrogen. The VGFARP peptide expression vectors obtained in this way can then be inserted into suitable cells, e.g. by electroporation. The VGFARP peptides produced in this way may be C- or N-terminally fused to heterologous sequences of peptides such as polyhistidine sequences, hemagglutinin epitopes (HAtag), or proteins such as, for example, maltose-binding proteins, glutathione S-transferase (GST), or protein domains such as the GAL-4 DNA binding domain or the GAL4 activation domain. The VGFARP peptides can be prepared by chemical synthesis for example in accordance with the Merrifield solid-phase synthesis protocol using automatic synthesizers which are obtainable from various manufacturers.

A further embodiment of this invention is the isolation of VGFARP peptides from biological samples or cell culture media or cell lysates from recombinant expression systems, e.g. using reverse phase chromatography, affinity chromatography, ion exchange chromatography, gel filtration, isoelectric focusing, or using other methods such as preparative immunoprecipitation, ammonium sulfate precipitation, extraction with organic solvents etc. A further embodiment of the invention is the obtaining of monoclonal or polyclonal antibodies using VGFARP peptides. The obtaining of antibodies takes place in the conventional way familiar to the skilled worker. A preferred embodiment of the production and obtaining of VGFARP peptide-specific antibodies, and a particularly preferred embodiment is the production of VGFARP peptide-specific antibodies which recognize neoepitopes, i.e. epitopes which are present only on VGFARP peptides but not in a VGF protein. Such anti-VGFARP peptide antibodies make the specific immunological detection of VGFARP peptides possible in the presence of VGF protein. Polyclonal antibodies can be produced by immunizations or experimental animals such as, for example, mice, rats, rabbits or goats. Monoclonal antibodies can be obtained for example by immunizations of experimental animals and subsequent application of hybridoma techniques or else via recombinant experimental approaches such as, for example, via antibody libraries such as the HuCAL® antibody library of MorphoSys, Martinsried, Germany, or other recombinant production methods known to the skilled worker. Antibodies can also be used in the form of antibody fragments such as, for example, Fab fragments or Fab2 fragments etc.

Therapy Development and Monitoring Through VGFARP Peptide Determinations

A further exemplary use is the quantitative or qualitative determination of the abovementioned VGFARP peptides or VGF proteins for estimating the efficacy of a therapy under development for neurological diseases, in particular chronic dementia diseases, in particular Alzheimer's disease. The invention can also be used to identify suitable patients for clinical studies for developing therapies for these diseases, in particular Alzheimer's disease. This entails comparison of quantitative measured results from a sample to be investigated with the measurements obtained in a control group and a group of patients. The efficacy of a therapeutic agent, or the suitability of the patient for a clinical study, can be inferred from these results. The testing of efficacy and the selection of the correct patients for therapies and for clinical studies is of outstanding importance for successful application and development of a therapeutic agent, and no clinically measurable parameter making this reliably possible is yet available for Alzheimer's disease [12].

Examination of the Therapeutic Efficacy of VGF Proteins, VGFARP Peptides and of Agents which Modulate the Expression and the Bioavailability of these Substances

One exemplary embodiment thereof encompasses the cultivation of cell lines and their treatment with VGF proteins, VGFARP peptides or with substances which promote the expression of VGF protein, such as, for example, NGF, BNDF or NT-3, or promote the processing of VGF protein to VGFARP peptides, such as, for example, prohormone convertases. It is possible thereby to establish the biological properties of VGF protein and VGFARP peptides in connection with neurological diseases, in particular Alzheimer's disease. Fusion proteins and fusion peptides can also be used for the treatment of the cell lines, e.g. fusion proteins consisting of prohormone convertases fused to peptide sequences which promote transport of the fusion protein into the interior of the cell. Examples of possible fusion partners of, for example, prohormone convertases are HIV TAT sequences or antennapedia sequences etc. It is likewise possible to transfect cell lines with expression vectors which bring about, directly or indirectly, expression of VGF protein or VGFARP peptides by the transfected cells. These expression vectors may code inter alia for VGFARP peptides, VGF proteins, NGF, BNDF, NT-3 or for prohormone convertases. Transfection of combinations of the said proteins can also be carried out. Alternatively, suitable cell lines can be treated with anti-VGF protein or anti-VGFARP peptide antibodies or with nucleic acids which suppress the expression of VGF, such as, for example, VGF antisense nucleic acids, VGF triplex nucleic acids or ribozymes directed against VGF mRNA. Treatment with anti-NGF, anti-BNDF or anti-NT-3 antibodies might also be carried out to suppress VGF protein expression. Cell lines which appear suitable as neurological model systems in connection with VGF in particular can be used for such investigations. Read-out systems which can be used for these investigations are inter alia tests which measure the rate of proliferation of the treated cells, their metabolic activity, the rate of apoptosis of the cells, changes in cell morphology, in the expression of cell-intrinsic proteins or reporter genes or which measure the release of cytosolic cell constituents as markers for cell death. Further test systems which can be used are suitable strains of experimental animals, e.g. of mice or rats, which are considered as model of neurological diseases, in particular as model of Alzheimer's disease. These experimental animals can be used to investigate the efficacy of therapeutic strategies which aim to modulate the concentration of VGFARP peptides or of VGF proteins. It is additionally possible to investigate proteins and peptides such as, for example, VGF proteins, VGFARP peptides, NGF, BNDF, NT-3, prohormone convertases etc. in experimental animals, it being possible for these peptides and proteins in some circumstances to be pharmaceutically processed so that they are better able to cross the blood-brain barrier and/or the blood-CSF barrier. It is possible to use as pharmaceutical processing method inter alia liposome-packaged proteins and peptides, proteins and peptides fused to transport sequences such as, for example, an HIV TAT sequence etc. In addition, peptides and proteins can be chemically modified in such a way that they acquire more lipophilic properties and are therefore able to penetrate more easily into cells. Peptides which are only slightly soluble in aqueous solutions can conversely be chemically modified so that they become more hydrophilic and then can be used for example as intravenously injectable therapeutic agent. Acid-resistant capsules can be used to protect sensitive substances, intended for oral administration, in the stomach.

Read-out parameters in experiments with animal models may be the survival time of the animals, their behavior and their short-term memory. One example of a memory test which is suitable for experimental animals is the Morris water maze test. Further parameters which can be used are the determination of body function such as, for example, blood tests, measurement of brain currents, metabolism test, the rate of expression of VGF protein and VGFARP peptides and other proteins associated with the disease, and morphological and histological investigations on tissues such as, for example, the brain.

Methods of Treatment

Another embodiment of the invention comprises methods of treatment of neurological diseases, in particular of chronic dementia diseases, like Alheimer disease, etc. At least one of the peptides, nucleic acids, antibodies, agonists or antagonists as defined herein may be used therein. The method may result in a reduction or increase, respectively, in the concentration of the altered VGFARP peptides or VGF proteins.

In particular, the method comprises administering a)antibodies directed against VGF proteins, VGFARP peptides, NGF, BNDF or NT-3 are administered, and/or b) antisense nucleic acids, triplex nucleic acids or ribozymes are administered, in order to reduce the expression of VGF proteins, VGFARP peptides, NGF, BNDF or NT-3, and/or c) substances which inhibit the processing of VGF proteins are administered, and/or d) antagonists of the VGFARP peptides or VGF protiens to a patient suffering from a neurological disease for a reduction of the concentration of VGFARP peptides. Alternatively, the method comprises administering to a patient suffering from a neurological disease for an increase of the concentration of VGFARP peptides a) VGF proteins, VGFARP peptides, NGF, BNDF or NT-3, and/or b) nucleic acids which code for VGF proteins, VGFARP peptides, NGF, BNDF or NT-3, and/or c) substances which promote the processing of VGF proteins, and/or d) agonists of the VGFARP peptides or of VGF proteis are administered to a patient.

The invention is illustrated in detail below by means of examples. Reference is also made to the figures in this connection.

FIG. 1 shows an alignment of the peptides of the invention with two known variants of the VGF protein which are identified in the figure by their database accession No. NM_{—}003378 (SEQ ID NO:44) and Y12661 (SEQ ID NO:43) . Sequence positions which are identical in both variants of the VGF proteins are represented by an asterisk in the sequence of NM_{—}003378 (SEQ ID NO:44). Different sequences are represented by the amino acid code in white letters on black background. The arrow at the end or at the start of partial sequences of VGFARP-12 (SEQ ID NO:10), -13 (SEQ ID NO:11), 45 (SEQ ID NO:42) and 34 (SEQ ID NO:31) indicates that the respective sequence extends over two lines in the alignment.

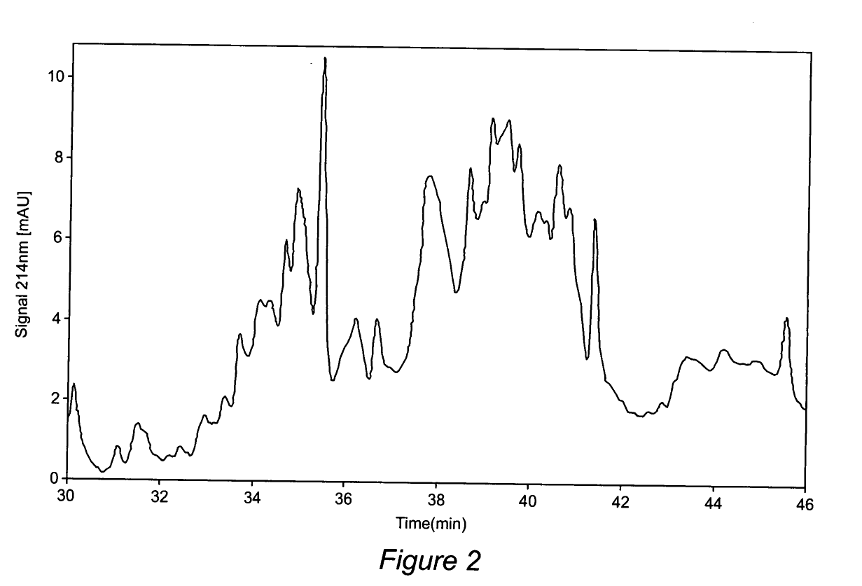

FIG. 2 shows a chromatogram recorded using reverse phase chromatography as in Example 2 for the separation and enrichment of the VGF peptides from cerebrospinal fluid.

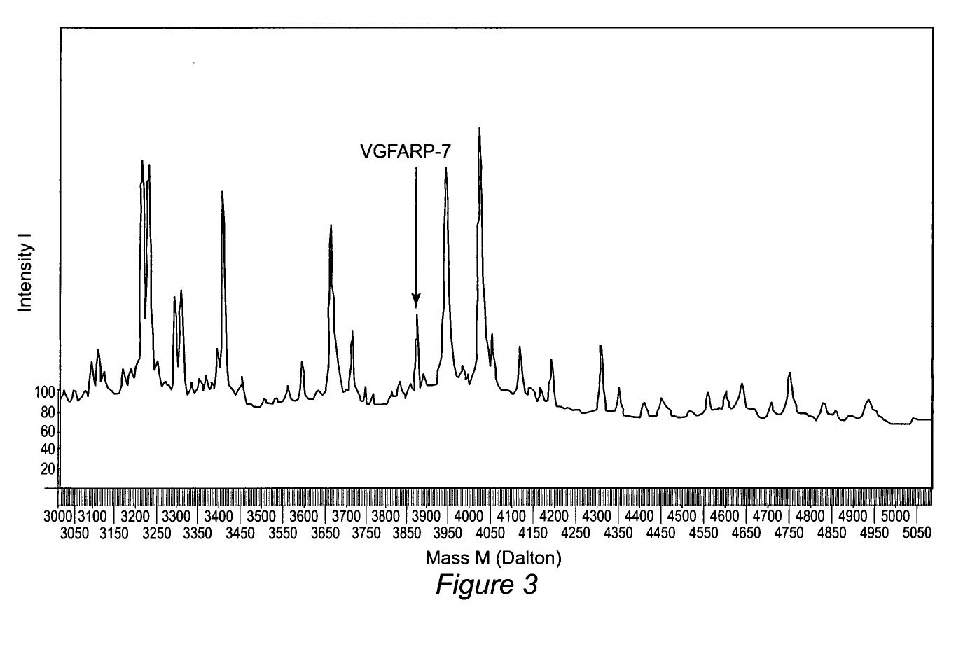

FIG. 3 shows a spectrum resulting from MALDI mass spectrometric measurement as in Example 3 of VGFARP-7 (SEQ ID NO:7), with a theoretical monoisotopic mass of 3686 dalton, after reverse phase chromatography of human cerebrospinal fluid as in Example 2. VGFARP-7 (SEQ ID NO:7) corresponds to the VGF sequence of Seq. ID 43 (accession No. Y12661) of amino acid 26-62.

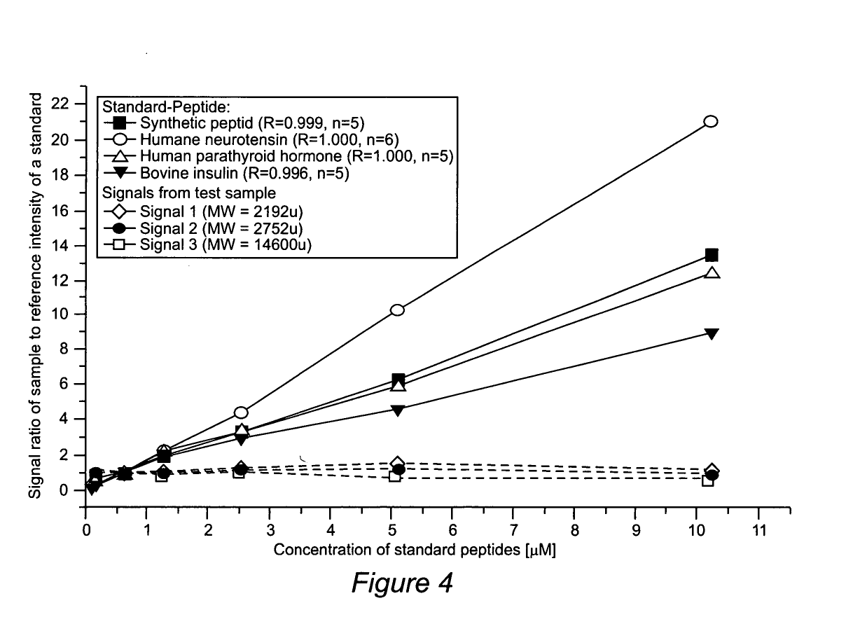

FIG. 4 shows data generated by MALDI as relatively quantifying MS method. A sample was mixed with various amounts of different standard peptides, and the intensity both of these standard signals and of representative sample signals was measured. All signal intensities of the standards were standardized to their signal intensity at a concentration of 0.64 μM (=1). Each peptide shows an individual typical ratio of signal strength to concentration, which can be read off in this diagram from the gradient of the plot.

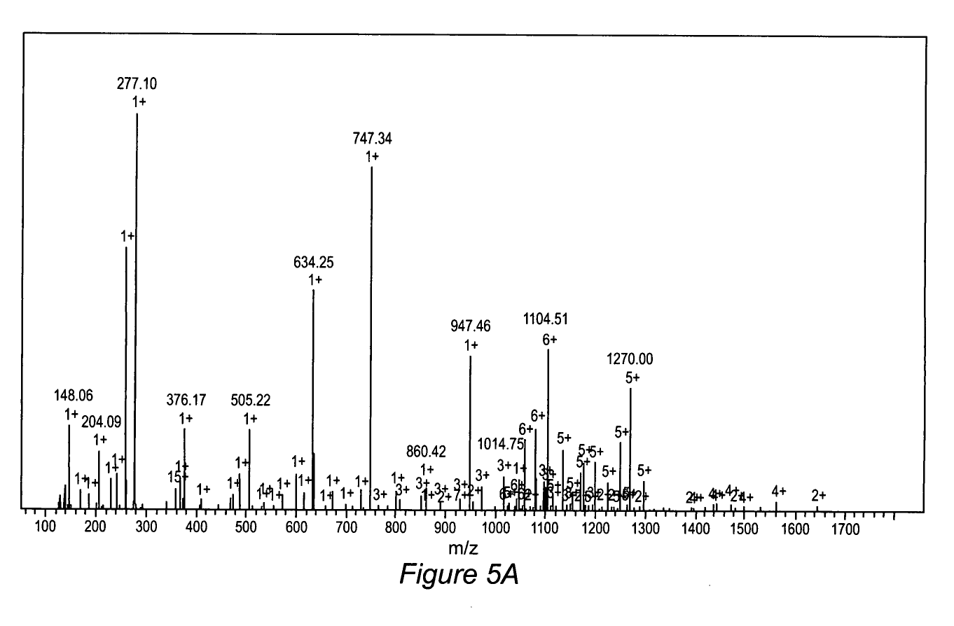

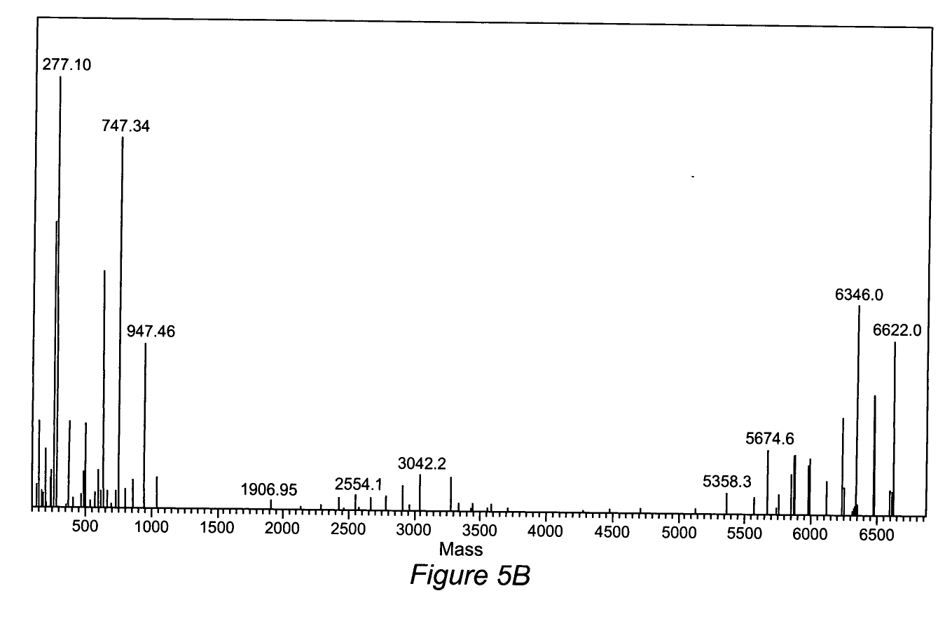

FIG. 5 shows an MS/MS fragment spectrum as in Example 4 of the peptide VGFARP-13 (SEQ ID NO:11) of the invention.

Upper trace: raw data of the measurement.

Lower trace: converted, deconvoluted mass spectrum of VGFARP-13.

The peak pattern is characteristic of VGFARP-13 (SEQ ID NO:11). VGFARP-13 (SEQ ID NO:11) corresponds to the VGF sequence of Seq. ID 43 (accession No. Y12661) of amino acid 421-479.

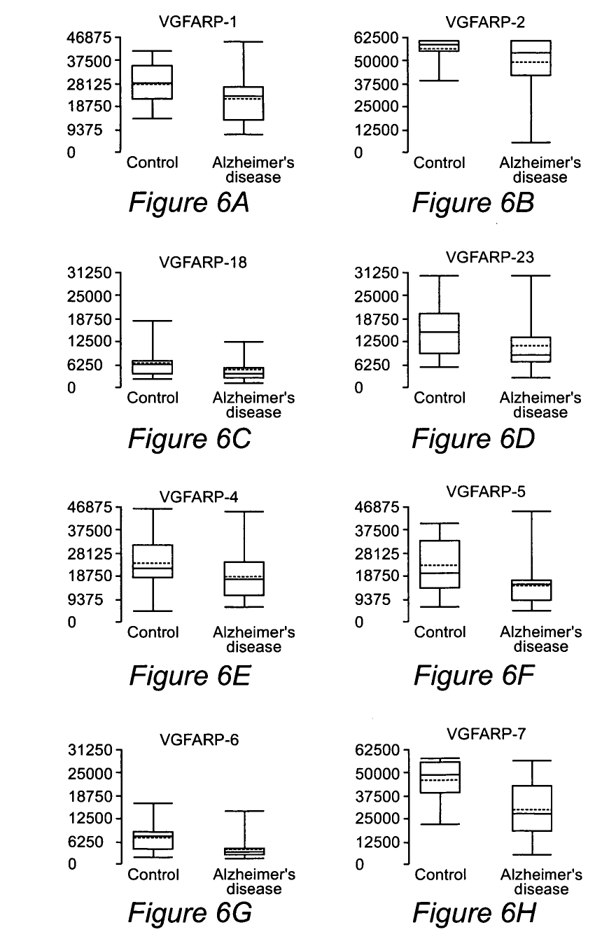

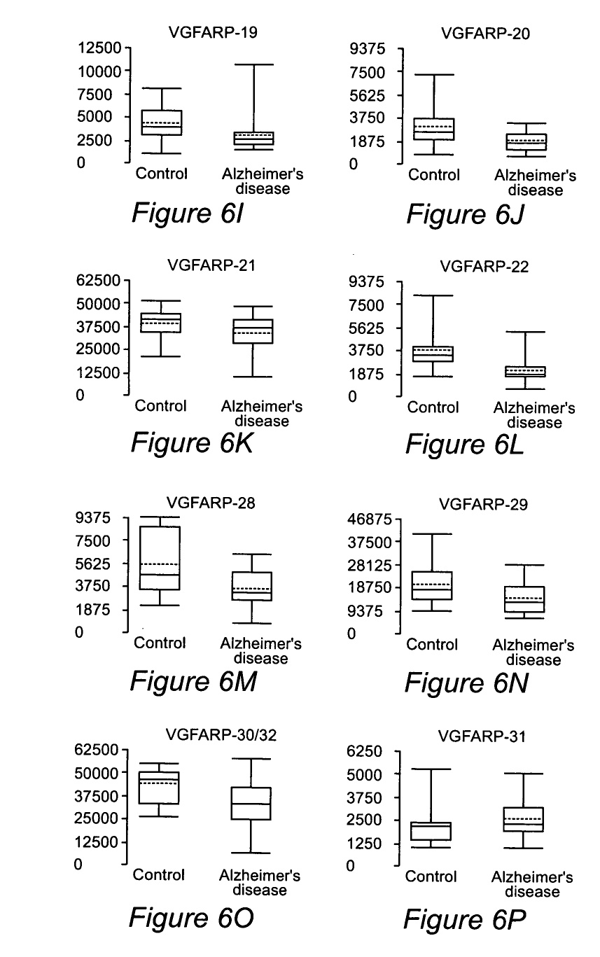

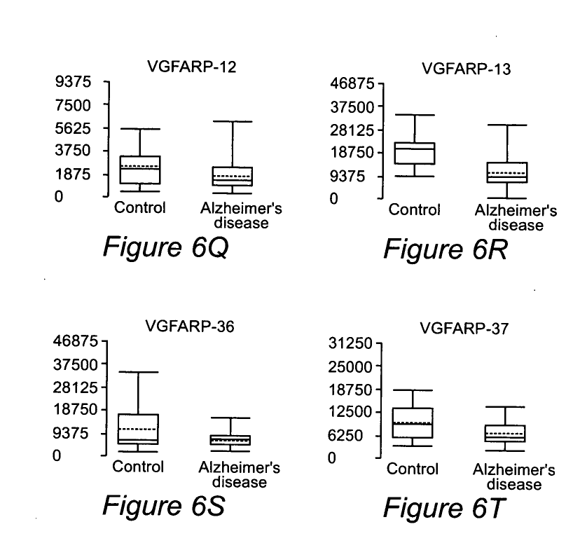

FIGS. 6A to 6C show in the form of box-whisker plots a comparison of the integrated MALDI mass spectrometric signal intensities of various VGFARP peptides in controls, compared with the signal intensities in samples from Alzheimer's disease patients.

### EXAMPLE 1

### Obtaining Cerebrospinal Fluid for Determining VGFARP Peptides

CSF or cerebrospinal fluid (fluid of the brain and spinal cord) is the fluid which is present in the four ventricles of the brain and in the subarachnoid space and which is produced in particular in the choroid plexus of the lateral ventricle. Cerebrospinal fluid is usually taken by lumbar puncture and less often by suboccipital puncture or ventricular puncture. In lumbar puncture (spinal puncture), to take cerebrospinal fluid, the puncture involves penetration of the spinal subarachnoid space between the 3rd and 4th or the 4th and 5th lumbar spinous process with a long hollow needle, and thus CSF being obtained. The sample is then centrifuged at 2000×g for 10 minutes, and the supernatant is stored at −80° C.

### EXAMPLE 2

### Separation of Peptides in Cerebrospinal Fluid (CSF) for Mass Spectrometric Measurement of VGFARP Peptides

For the detection of VGF peptides in CSF by mass spectrometry, it is necessary in this example to separate the peptide constituents. This sample pretreatment serves to concentrate the peptides of the invention and to remove components which may interfere with the measurement. The separation method carried out is a reverse phase chromatography. Various RP chromatography resins and eluants are equally suitable for this. The separation of VGF peptides using a C18 reverse phase chromatography column with the size of 4 mm×250 mm supplied by Vydac is [lacuna] by way of example below. Mobile phases of the following composition were used: mobile phase A: 0.06% (v/v) trifluoroacetic acid, mobile phase B: 0.05% (v/v) trifluoroacetic acid, 80% (v/v) acetonitrile. Chromatography took place at 33° C. using an HP ChemStation 1100 supplied by Agilent Technologies with a micro flow cell supplied by Agilent Technologies. Human cerebrospinal fluid was used as sample. 440 μl of CSF were diluted with water to 1650 μl, the pH was adjusted to 2-3, the sample was centrifuged at 18 000× for 10 minutes and finally 1500 μl of the sample prepared in this way were loaded onto the chromatography column. The chromatography conditions were as follows: 5% mobile phase B at time 0 min, from time 1 to 45 min continuous increase in the mobile phase B concentration to 50%, from time 45 to 49 min continuous increase in the mobile phase B concentration to 100% and subsequently up to time 53 min constant 100% buffer B. Collection of 96 fractions each of 0.5 ml starts 10 minutes after the start of the chromatography. The chromatogram of a cerebrospinal fluid sample prepared under the experimental conditions described herein is depicted in FIG. 2.

### EXAMPLE 3

### Measurement of Masses of Peptides by Means of MALDI Mass Spectrometry

For mass analysis, typical positive ion spectra of peptides were produced in a MALDI-TOF mass spectrometer (matrix-assisted laser desorption ionization). Suitable MALDI-TOF mass spectrometers are manufactured by PerSeptive Biosystems Framingham (Voyager-DE, Voyager-DE PRO or Voyager-DE STR) or by Bruker Daltonik Bremen (BIFLEX). The samples are prepared by mixing them with a matrix substance which typically consists of an organic acid. Typical matrix substances suitable for peptides are 3,5-dimethoxy-4-hydroxycinnamic acid, α-cyano-4-hydroxycinnamic acid and 2,5-dihydroxybenzoic acid. A lyophilized equivalent obtained by reverse phase chromatography and corresponding to 500 μl of human cerebrospinal fluid is used to measure the VGFARP peptides of the invention. The chromatographed sample is dissolved in 15 μl of a matrix solution. This matrix solution contains, for example, 10 g/l α-cyano-4-hydroxycinnamic acid and 10 g/l L(-)fucose dissolved in a solvent mixture consisting of acetonitrile, water, trifluoroacetic acid and acetone in the ratio 49:49:1:1 by volume. 0.3 μl of this solution is transferred to a MALDI carrier plate, and the dried sample is analyzed in a Voyager-DE STR MALDI mass spectrometer from PerSeptive Biosystems. The measurement takes place in linear mode with delayed extraction™. An example of a measurement of one of the VGFARP peptides of the invention is shown in FIG. 3.

The MALDI-TOF mass spectrometer can be employed to quantify peptides such as, for example, the VGFARP peptides of the invention if these peptides are present in a concentration which is within the dynamic measurement range of the mass spectrometer, thus avoiding detector saturation. This is the case for the measurement of the VGFARP peptides of the invention in cerebrospinal fluid at a CSF equivalent concentration of 33.3 μl per μl of matrix solution. There is a specific ratio between measured signal and concentration for each peptide, which means that the MALDI mass spectrometry can preferably be used for the relative quantification of peptides. This situation is depicted in FIG. 4. If various amounts of different standard peptides are added to a sample, it is possible to measure the intensity both of these standard signals and of the sample signals. FIG. 4 shows by way of example a MALDI measurement as relatively quantifying MS method. All signal intensities of the standards were standardized to their signal intensity at a concentration of 0.64 μM (=1). Each peptide shows an individual, typical ratio of signal strength to concentration, which can be read off from the gradient of the plot.

### EXAMPLE 4

### Mass Spectrometric Identification of the VGFARP Peptides

For quantification of the VGFARP peptides of the invention it is necessary to ensure that the mass signals to be analyzed of peptides in the fractions obtained by reverse phase chromatography of cerebrospinal fluid, as in Example 2, in fact relate to the VGFARP peptides of the invention.

The peptides of the invention are employed in these fractions for example using nanoSpray-MS/MS [11]. This entails a VGFARP peptide ion in the mass spectrometer being selected in the mass spectrometer on the basis of its specific m/z (mass/charge) value in a manner known to the skilled worker. This selected ion is then fragmented by supplying collisional energy with an impinging gas, e.g. helium or nitrogen, and the resulting VGFARP peptide fragments are detected in the mass spectrometer in an integrated analysis unit, and corresponding m/z values are determined (principle of tandem mass spectrometry) [13]. The fragmentation behavior of peptides makes unambiguous identification of the VGFARP peptides of the invention possible when the accuracy of mass is, for example, 50 ppm by the use of computer-assisted search methods [14] in sequence databases into which the sequence of a VGF protein has been entered. In this specific case, the mass spectrometric analysis took place with a Quadrupol-TOF Instrument, QStar-Pulsar model from Applied Biosystems-Sciex, USA. Examples of MS/MS fragment spectra are shown in FIG. 5.

### EXAMPLE 5

### Mass Spectrometric Quantification of the VGFARP Peptides to Compare Their Relative Concentration in Control Samples Compared with Patients' Samples

A sample preparation as in Example 1 and 2 followed by a MALDI measurement of the VGFARP peptides of the invention as in Example 3 were carried out on 222 clinical samples, i.e. 82 control samples and 130 samples from patients suffering from Alzheimer's disease. Examples of MALDI signal intensities are depicted in the form of box-whisker plots in FIGS. 6A to 6C. The box-whisker plots depicted in FIG. 6 are based on measurements carried out in each case on 29 to 45 samples from Alzheimer's disease patients, and 13 to 44 control samples per experiment. A total of 4 experiments was carried out. The box-whisker plots depicted make it possible to compare the integrated MALDI mass spectrometric signal intensities of various VGFARP peptides in controls with the MALDI signal intensities in samples from Alzheimer's disease patients. In these, the box, i.e. the columns in the diagrams in FIGS. 6A to 6C, in each case includes the range of MALDI signal intensities in which 50% of the respective MALDI signal intensities are to be found, and the lines starting from the box and pointing upward and downward (whiskers) indicate the range in which in each case the 25% of measurements which show the highest signal intensities (upper quarter) are to be found, and in which the 25% of measurements which show the lowest signal intensities (lower quarter) are to be found. The full line in the columns indicates the median and the broken line in the columns indicates the mean.

The headings in this document are intended merely to provide structure to the text. They are not intended to limit or restrict the matters described. All the examples are intended to characterize the concept of the invention in more detail but are not intended to restrict the equivalence range of the invention.

We claim:

1. A method for detecting a chronic dementia disease or a predisposition to a chronic dementia disease in a patient in need thereof, comprising the steps of
obtaining a biological sample from said patient,
determining a concentration of at least one VGF protein or VGFARP peptide in the biological sample, and
comparing the concentration of the at least one VGF protein or VGFARP peptide in the biological sample to the concentration of the same protein or peptide in a control sample, wherein a difference between the concentration of the VGF protein or VGFARP peptide in the biological sample compared to the concentration of the VGF protein or VGFARP peptide in the control sample is indicative of chronic dementia disease or a predisposition to a chronic dementia disease.

2. The method of claim 1, wherein the at least one VGF protein or VGFARP peptide is selected from the group consisting of: SEQ ID NO:43; SEQ ID NO:44;a mutant of SEQ ID NO:43 in which the amino acid sequence of the mutant differs by a maximum of 20% from the amino acid sequence of SEQ ID NO:43; a mutant of SEQ ID NO:44 in which the amino acid sequence of the mutant differs by a maximum of 20% from the amino acid sequence of SEQ ID NO:44; a protein that represents a naturally occurring allele of a VGF protein; a peptide derivate derived from a naturally occurring allele of a VGF protein; a peptide derivate derived from a VGFARP peptide; a VGFARP peptide mutant that differs by a maximum of 2 amino acids from the corresponding unmutated VGFARP peptide.

3. The method of claim 2, wherein said VGFARP peptide is selected from the group consisting of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, and SEQ ID NO:42.

4. The method of claim 2 wherein the at least one VGF protein or VGFARP peptide is chemically modified.

5. The method of claim 2 wherein the at least one VGF protein or VGFARP peptide is post-translationally modified.

6. The method of claim 1 wherein said method is carried out in combination with other diagnostic methods for chronic dementia diseases to increase the sensitivity and/or specificity thereof.

7. The method of claim 1 wherein the dementia disease is selected from the group consisting of Alzheimer's disease or a related neurological disease; Lewy body dementia; and vascular dementia.

8. The method of claim 1 wherein for a positive detection of the disease the concentration of the at least one VGF protein or VGFARP peptide is raised or lowered relative to the concentration of the VGF protein or VGFARP peptide in a control sample.

9. The method of claim 1 wherein the method is used to determine a parameter selected from the group consisting of: the severity of the disease, prognosis of the course of the disease, diagnosis of preliminary stages of neurological diseases, and mild cognitive impairment (MCI).

10. The method of claim 1 wherein the biological sample is selected from the group consisting of cerebrospinal fluid, serum, plasma, urine, synovial fluid, stool, tear fluid, sputum and a tissue homogenate.

11. The method of claim 1 wherein the at least one VGF protein or VGFARP peptide is identified by mass spectrometry.

12. The method of claim 11, wherein identification of the at least one VGF protein or VGFARP peptide by mass spectroscopy includes the determination of at least one of the theoretical monoisotopic mass peaks selected from the group consisting of 3666.8278/3950.9875/3567.7594/3595.7907/3879.9504/3401.6852/3614.8077/3685.8448/3302.6167/3173.5741/3955.9889/1336.6735/2503.1827/≧727.3501/≧851.4137/≧730.3246/3745.7343/1235.5782/≧833.4395/7518.2744/2031.8981/2418.0419/4806.0408/3456.5513/4806.0408/4058.7043/5776.6294/6618.0363/1380.7249/≧946.4468/≧862.3192/≧961.4063/3903.0180/3787.9911/≧920.4828/656.3242/3782.8976/1886.8970/1672.7653/≧792.3501/3343.4672 and 2220.1889 dalton.

13. The method of claim 1, wherein the at least one VGF protein or VGFARP peptide is identified with an immunological test.

14. The method of claim 13, wherein said immunological test is selected from the group consisting of enzyme linked immuno sorbent assay (ELISA), a radioimmunoassay, and a Western blot.

15. The method of claim 13, wherein the at least one VGF protein or VGFARP peptide is identified using a substance that binds to the protein or peptide.

16. The method of claim 15, wherein the substance that binds to the at least one VGF protein or VGFARP peptide is selected from the group consisting of an antibody, an antibody fragment, a phage particle, and an affinity matrix.

17. The method of claim 1 further comprising the step of chromatographically fractionating said biological sample prior to said determining step.

18. The method of claim 17 wherein said step of chromatographically fractionating said biological sample is carried out using reverse phase chromatography or high resolution reverse phase chromatography.

19. The method of claim 1 further comprising the step of fractionating the biological sample by precipitation reactions or liquid phase separations prior to said determining step.

20. The method of claim 1 wherein said step of determining is carried out using antibodies against at least one VGF protein or VGFARP peptide.

21. The method of claim 1 wherein said step of determining is carried out by detection of nucleic acids encoding at least one VGF protein or VGFARP peptide.

22. The method of claim 21 wherein the detection of nucleic acids is carried out using Northern blots, reverse transcriptase PCR or quantitative PCR.

23. A method for diagnosing a neurological disease in a patient, comprising the step of
obtaining a biological sample from said patient,
determining a concentration of at least one VGF protein or VGFARP peptide in the biological sample, and
comparing the concentration of the at least one VGF protein or VGFARP peptide in the biological sample to the concentration of the same protein or peptide in a control sample, wherein a difference between the concentration of the VGF protein or VGFARP peptide in the biological sample compared to the concentration of the VGF protein or VGFARP peptide in the control sample is indicative of a neurological disease.

24. The method of claim 23 wherein said step of determining is carried out using antibodies against at least one VGF protein or VGFARP peptide.

25. The method of claim 23 wherein said step of determining is carried out by detection of nucleic acids encoding at least one VGF protein or VGFARP peptide.

26. The method of claim 25 wherein the detection of nucleic acids is carried out by using Northern blots, reverse transcriptase PCR or quantitative PCR.

27. The method of claim 23, wherein the method is used to monitor the efficacy of a therapy for a neurological disease.

28. The method of claim 23, wherein the method is used for stratifying patients who are suitable for therapies or clinical studies of neurological diseases.

29. The method of claim 23, wherein the neurological disease is selected from the group consisting of chronic dementia disease and Alzheimer's disease.

30. A method for prophylaxis or treatment of a neurological disease in a patient in need thereof, comprising the step of
administering to the patient a substance that causes modulation of the concentration of at least one VGF protein or VGFARP peptide in a quantity sufficient to prevent or treat the neurological disease.

31. The method of claim 30, wherein the neurological disease is selected from the group consisting of chronic dementia disease and Alzheimer's disease.

32. The method of claim 30, wherein the modulation is a reduction in concentration of the at least one VGF protein or VGFARP peptide.

33. A method of claim 30, wherein the modulation is an increase in concentration of the at least one VGF protein or VGFARP peptide.

34. The method of claim 30, wherein the substance is selected from the group consisting of:
a) antibodies directed against VGF proteins, VGFARP peptides, NGF, BNDF or NT-3;
b) antisense nucleic acids, triplex nucleic acids or ribozymes that reduce expression of VGF proteins, VGFARP peptides, NGF, BNDF or NT-3;
c) substances that inhibit processing of VGF proteins; and
d) antagonists of VGFARP peptides or VGF proteins.

35. The method of claim 30, wherein the substance is selected from the group consisting of:
a) VGF proteins, VGFARP peptides, NGF, BNDF or NT-3;
b) nucleic acids which code for VGF proteins, VGFARP peptides, NGF, BNDF or NT-3;
c) substances which promote the processing of VGF proteins, and
d) agonists of the VGFARP peptides or of VGF proteins.

36. The method of claim 30, wherein the substance modulates the expression of at least one VGF protein.

37. The method of claim 36 wherein the VGF protein is selected from the group consisting of NGF, BNDF and NT-3.

38. The method of claim 30, wherein the substance selectively inhibits or stimulates the transcription or expression of at least one VGF protein.

39. The method of claim 30, wherein the substance binds to at least one VGF protein or VGFARP peptide.

40. The method of claim 39 wherein the substance is selected from the group consisting of antibodies, antibody fragments, and affinity matrices.

41. The method of claim 30 wherein the substance is administered via an administration route selected from the group consisting of: the bloodstream, the gastrointestinal tract, the urogenital tract, the lymphatic system, the subarachnoid space, the lungs, and direct injection into tissue.

42. The method of claim 41, wherein the tissue is selected from the group consisting of muscle tissue, adipose tissue, and brain tissue.

43. The method of claim 30, wherein the substance has been pharmaceutically processed or chemically or biologically modified to cross the blood-brain barrier and/or the blood-CSF barrier.

44. A VGFARP peptide.

45. The VGFARP peptide of claim 44, wherein the VGFARP peptide is a derivative of a VGF protein.

46. The VGFARP peptide of claim 45, wherein the VGFARP peptide is a derivative of a VGF allele.

47. The VGFARP peptide of claim 44, wherein the VGFARP peptide is a mutant VGFARP peptide that differs by a maximum of 2 amino acids from the corresponding unmutated VGFARP peptide.

48. The VGFARP peptide of claim 44, wherein the sequence of the VGFARP peptide is selected from the group consisting of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, and SEQ ID NO:42.

49. The VGFARP peptide of claim 45, wherein said VGFARP peptide is a derivative of a VGF protein represented by SEQ ID NO:43 or SEQ ID NO:44.

50. The VGFARP peptide of claim 44, wherein said peptide is chemically or post-translationally modified.

51. A nucleic acid molecule that encodes a VGFARP peptide.

52. A nucleic acid molecule that is the complement of a nucleic acid molecule that encodes a VGFARP peptide.

53. A pharmaceutical composition comprising,
at least one VGF protein or VGFARP peptide, a nucleic acid encoding at least one VGF protein or VGFARP peptide, or a nucleic acid that is the complement of a nucleic acid encoding at least one VGF protein or VGFARP peptide.

54. A diagnostic reagent for the detection of neurological diseases, comprising,
antibodies to at least one VGF protein or VGFARP peptide, and
a suitable carrier.

55. The diagnostic reagent of claim 54, wherein the neurological disease is selected from the group consisting of a neurological disease, chronic dementia, and Alzheimer's disease.

56. Antibodies that bind to VGFARP peptides.

57. Nucleic acids that are VGF-specific antisense nucleic acids, components of VGF-specific ribozymes, or VGF-specific triplex nucleic acids.
